(19) United States
(12) Patent Application Publication
(10) Pub. No.: US 2025/0281042 A1
(43) Pub. Date: Sep. 11, 2025

(54) BIOLOGICAL INFORMATION OUTPUT DEVICE

(71) Applicant: Murata Manufacturing Co., Ltd., Nagaokakyo-shi (JP)

(72) Inventors: Takaya SANO, Nagaokakyo-shi (JP); Shuto Kimura, Nagaokakyo-shi (JP); Koji Tanaka, Nagaokakyo-shi (JP)

(21) Appl. No.: 19/220,641

(22) Filed: May 28, 2025

Related U.S. Application Data
(63) Continuation of application No. PCT/JP2023/042489, filed on Nov. 28, 2023.

(30) Foreign Application Priority Data
Nov. 29, 2022 (JP) 2022-190488

(51) Int. Cl. A61B 5/00; H02J 7/00

(52) U.S. Cl. CPC A61B 5/0002; H02J 7/00032; H02J 7/0048; A61B 2505/05; A61B 2560/0204

(57) ABSTRACT

A CPU performs an initial-battery-level acquiring process. In the initial-battery-level acquiring process, the CPU acquires the initial battery level which is the level of a battery at start of running of a biological information output device. The CPU performs a first level-determination process at start of running. In the first level-determination process at start of running, the CPU determines whether the initial battery level is less than or equal to a first level. The first level is the level of a battery required for the biological information output device to run just for a preset time which is predetermined. If the CPU determines that the initial battery level is less than or equal to the first level, the CPU performs a notification process. In the notification process, the CPU outputs a notification signal.

### CROSS REFERENCE TO RELATED APPLICATION

This is a continuation of International Application No. PCT/JP2023/042489 filed on Nov. 28, 2023 which claims priority from Japanese Patent Application No. 2022-190488 filed on Nov. 29, 2022. The contents of these applications are incorporated herein by reference in their entireties.

### BACKGROUND OF THE DISCLOSURE

### Field of the Disclosure

The present disclosure relates to a biological information output device.

### Description of the Related Art

Japanese Unexamined Patent Application Publication No. 2015-141080 describes a battery-powered device. The device includes power-supply-voltage monitoring means that detects the battery level. In reset of the device due to battery consumption, the power-supply-voltage monitoring means stores a flag indicating the reset. At the next startup of the device, the power-supply-voltage monitoring means determines whether the battery has run out of power on the basis of the battery level and whether the flag has been stored.

### BRIEF SUMMARY OF THE DISCLOSURE

A biological information output device like the device described in Japanese Unexamined Patent Application Publication No. 2015-141080 runs on power supply from a battery. The biological information output device outputs biological information. The biological information output device may stop at an unexpected time due to occurrence of battery depletion during running. In particular, in the case of a biological information output device, a surgical operation may be performed by using biological information which is outputted from the biological information output device. During such a surgical operation, when the biological information output device runs out of battery power, outputting no biological information makes it difficult to continue the surgical operation.

To solve the issue, an aspect of the present disclosure provides a biological information output device comprising: a control unit that controls running and stopping of the biological information output device which runs on power supply from a battery and which outputs biological information. The control unit performs an initial-battery-level acquiring process of acquiring an initial battery level which is the level of the battery at start of the running of the biological information output device, a first level-determination process of determining whether the initial battery level is less than or equal to a first level, the first level being a level required for the biological information output device to run just for a preset time which is predetermined, and a notification process of, when it is determined that the initial battery level is less than or equal to the first level in the first level-determination process, outputting a notification signal for notifying a user that the running will not continue for the preset time.

The configuration enables a user to confirm, on the basis of whether the notification has been received, if it is possible for the biological information output device to run for the preset time or longer from start of the running. This suppresses occurrence of an unexpected stop of the biological information output device in the preset time.

Occurrence of an unexpected stop of the biological information output device may be suppressed.

### BRIEF DESCRIPTION OF THE SEVERAL VIEWS OF THE DRAWINGS

FIG. 1 is a schematic view of a biological information acquiring system according to an embodiment.

FIG. 2 is a block diagram illustrating a biological information output device.

FIG. 3 is a flowchart of a series of processes related to the battery level at start of running.

FIG. 4 is a flowchart of a series of processes related to the battery level during running.

FIG. 5 is a flowchart of a series of processes related to wireless communication.

FIG. 6 is a flowchart of a series of processes related to the continuous running time during running.

### DETAILED DESCRIPTION OF THE DISCLOSURE

### An Embodiment

A biological information output device according to an embodiment will be described below by referring to the drawings. In the description below, a biological information acquiring system including the biological information output device will be described as an example.

### <Biological Information Acquiring System>

As illustrated in FIG. 1, a biological information acquiring system 10 includes a biosensor 20, a biological information output device 30, and a display 40.

The biosensor 20 is attached on a monitoring target for use. For example, the biosensor 20 is attached on a monitoring target with an attachment sheet 21 interposed in between. The biosensor 20 monitors biological information, for example, of an inpatient or a person under surgery, who is a monitoring target. Specifically, examples of biological information include body temperature, blood pressure, blood glucose level, oxygen saturation, heart rate, and pulse rate. In the present embodiment, the biosensor 20 is capable of detecting multiple types of biological information.

The biosensor 20 is capable of wireless communication with another device. The biosensor 20 may transmit, to the biological information output device 30 through wireless communication, a signal indicating detected biological information. The system of the wireless communication is, for example, Bluetooth®.

The biological information output device 30 receives, from the biosensor 20 through wireless communication, the signal indicating biological information. Thus, the biological information output device 30 obtains, from the biosensor 20, monitoring results of biological information.

The biological information output device 30 includes a body case 31, an output terminal 32, and a small monitor 33. The body case 31 includes the units described below, and a battery 36 therein. The output terminal 32 may be connected to the display 40. The small monitor 33 is attached on the outer surface of the body case 31. The small monitor 33 displays an image smaller than one displayed on the display 40 described below.

The biological information output device 30 is capable of communicating with the display 40 through the output terminal 32 connected to the display 40. The biological information output device 30 outputs, through the output terminal 32 to the display 40, the obtained monitoring results of biological information.

The display 40 obtains, from the biological information output device 30, monitoring results of biological information. The display 40 displays, as an image indicating time-series data, the obtained monitoring results of biological information.

### <Biological Information Output Device>

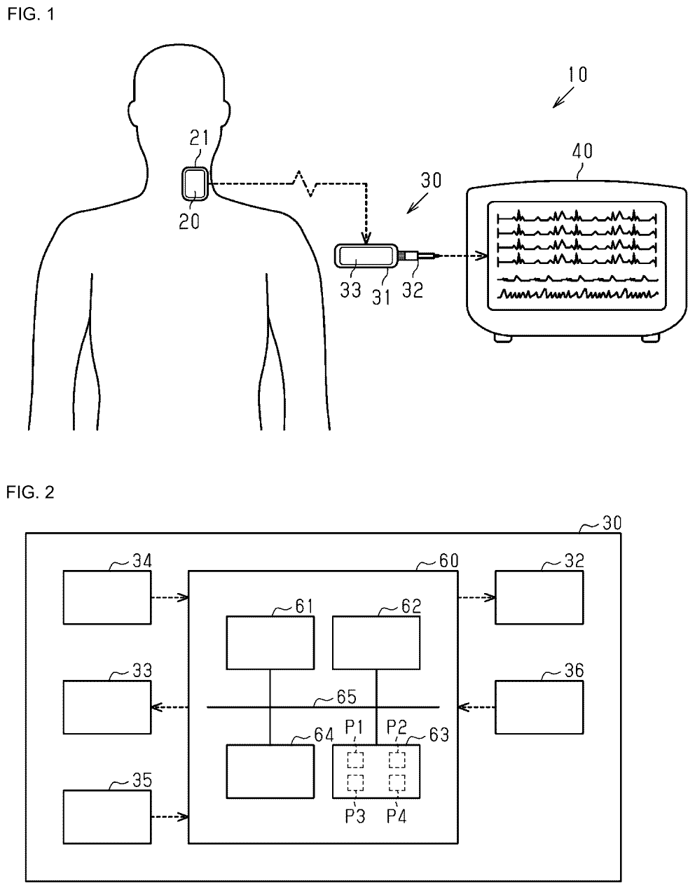

As illustrated in FIG. 2, the biological information output device 30 further includes a switch 34, a wireless receiver 35, the battery 36, and a control unit 60 in addition to the output terminal 32 and the small monitor 33, which are described above.

The switch 34 is used to switch the power supply of the biological information output device 30 between the ON state and the OFF state. For example, when the biological information output device 30 is in the OFF state, the switch 34 receiving a long-press operation causes the biological information output device 30 to enter the ON state. Thus, the biological information output device 30 starts running. When the biological information output device 30 is in the ON state, the switch 34 receiving a predetermined operation causes the control unit 60 to receive a preset signal in accordance with the operation. Examples of predetermined operations of the switch 34 include a long-press operation, a short-press operation, and a continuous-double-press operation. In the embodiment, when the biological information output device 30 is in the ON state, the switch 34 receiving a long-press operation causes the biological information output device 30 to enter the OFF state. The switch 34 is positioned on an outer surface of the body case 31 (not illustrated). For example, a user of the biological information output device 30 is a health professional.

The wireless receiver 35 receives a signal from the biosensor 20 through wireless communication. That is, the wireless receiver 35 receives, from the biosensor 20 through wireless communication, monitoring results of biological information. The wireless receiver 35 outputs, to the control unit 60, the received monitoring results of biological information.

The battery 36 is included in the body case 31. The battery 36 supplies power to the biological information output device 30. In the present embodiment, the battery 36 is a chargeable secondary battery. The biological information output device 30 runs on power supply from the battery 36.

The control unit 60 controls the running and stopping of the biological information output device 30. The control unit 60 includes a CPU 61, peripheral circuitry 62, a ROM 63, a storage device 64, and an internal bus 65. The internal bus 65 communicatively connects the CPU 61, the peripheral circuitry 62, the ROM 63, and the storage device 64 to one another. The peripheral circuitry 62 includes a circuit which generates a clock signal for regulating internal operations, a power supply circuit, and a reset circuit. The ROM 63 stores various programs in advance. The CPU 61 executes various programs, which are stored in the ROM 63, to control the biological information output device 30.

The ROM 63 stores an initial running program P1, a during-running program P2, a wireless communication program P3, and a continuous running program P4.

The storage device 64 may store, as a communication destination, the biosensor 20 with which the biological information output device 30 is wirelessly communicating. Specifically, the storage device 64 may store the unique ID of the biosensor 20 as a communication destination. In this embodiment, the storage device 64 may store only a single biosensor 20 which is a communication destination.

### <A Series of Processes Related to the Battery Level at Start of Running>

When a long-press operation on the switch 34 causes the biological information output device 30 to enter the ON state from the OFF state, that is, when the biological information output device 30 starts running, the CPU 61 executes the initial running program P1 only once.

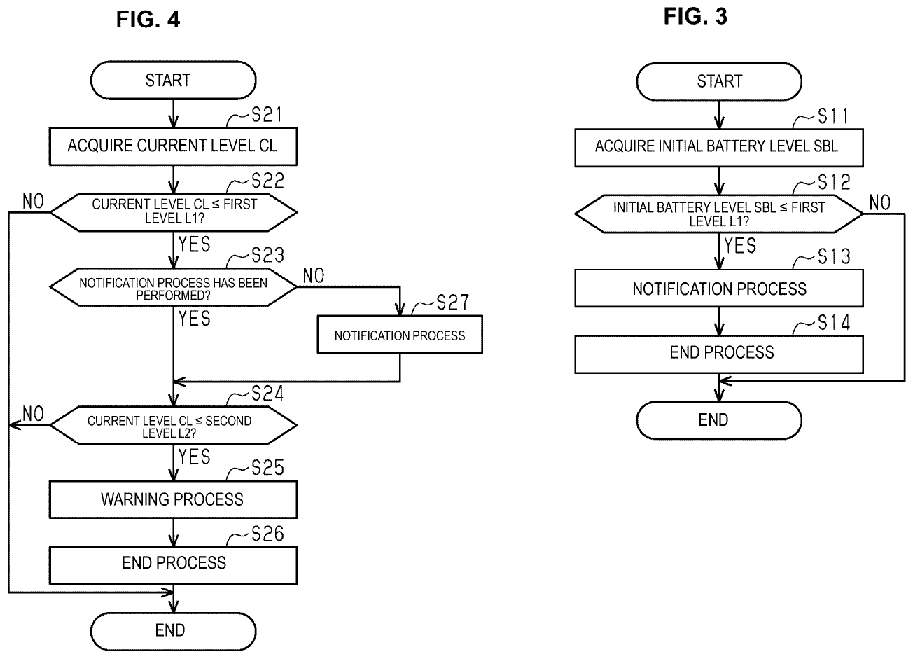

As illustrated in FIG. 3, at start of the initial running program P1, the CPU 61 first performs the process in step S11. In step S11, the CPU 61 performs an initial-battery-level acquiring process. In the initial-battery-level acquiring process, the CPU 61 acquires the initial battery level SBL which is the level of the battery 36 at start of running of the biological information output device 30. The CPU 61 calculates the level of the battery 36 by using a known method, for example, on the basis of the voltage across the terminals of the battery 36 or the integrated current value of the battery 36. After that, the CPU 61 causes the process to proceed to step S12.

In step S12, the CPU 61 performs a first level-determination process at start of running. In the first level-determination process at start of running, the CPU 61 determines whether the initial battery level SBL is less than or equal to a first level L1. The first level L1 is determined, through a test or a simulation, as the required level of the battery 36 for running the biological information output device 30 for a preset time RT which is determined in advance. The preset time RT is, for example, 12 hours.

Thus, the first level L1 does not indicate that the level of the battery 36 is quite low nor that the voltage of the battery 36 is unstable. That is, the first level L1 is a threshold for guaranteeing stable running of the biological information output device 30 for an appropriate time. Therefore, assume that the level of the battery 36 in the full-charge state is set to 100% and that a value of 30% or more is set to the first level L1. In this case, the first level L1 may be a level of the battery 36 which is required to run the biological information output device 30 for the preset time RT.

In step S12, if the CPU 61 determines that the initial battery level SBL is greater than the first level L1, (S12: NO), the CPU 61 ends the series of processes this time. In contrast, if the CPU 61 determines that the initial battery level SBL is less than or equal to the first level L1 (S12: YES), the CPU 61 causes the process to proceed to step S13.

In step S13, the CPU 61 performs a notification process. In the notification process, the CPU 61 outputs a notification signal. The notification signal is a signal for notifying a user that the biological information output device 30 will not run for the preset time RT. Specifically, in the notification process, the CPU 61 outputs the notification signal to the small monitor 33. Thus, the small monitor 33 displays a message image indicating that the biological information output device 30 will not run for the preset time RT. The message image is displayed for several seconds to more than ten seconds. After that, the CPU 61 causes the process to proceed to step S14.

In step S14, the CPU 61 performs an end process. In the end process, the CPU 61 first outputs a prior-notice-of-stop signal. The prior-notice-of-stop signal is a signal for providing, to a user, a prior notice of stopping the biological information output device 30. Specifically, in the end process, the CPU 61 outputs the notification signal to the small monitor 33. Thus, the small monitor 33 displays a message image indicating that the running of the biological information output device 30 will stop soon. The message image is displayed for several seconds to more than ten seconds. Then, the CPU 61 ends the running of the biological information output device 30 which is performed due to power supply from the battery 36. That is, the CPU 61 stops the biological information output device 30. Thus, the CPU 61 ends the series of processes this time.

### <A Series of Processes Related to the Battery Level During Running>

After the CPU 61 ends execution of the initial running program P1, if the biological information output device 30 continues to run, the CPU 61 repeatedly executes the during-running program P2 at predetermined intervals until the biological information output device 30 stops running.

As illustrated in FIG. 4, when the CPU 61 starts the during-running program P2, the CPU 61 first performs the process in step S21. In step S21, the CPU 61 performs a current-level acquiring process. In the current-level acquiring process, the CPU 61 acquires the current level CL which is the current level of the battery 36. After that, the CPU 61 causes the process to proceed to step S22.

In step S22, the CPU 61 performs the first level-determination process during running. In the first level-determination process during running, the CPU 61 determines whether the current level CL acquired in the current-level acquiring process in step S21 is less than or equal to the first level L1.

If the CPU 61 determines that the current level CL is greater than the first level L1 (S22: NO), the CPU 61 ends the series of processes this time. That is, the CPU 61 continues running the biological information output device 30. In contrast, if the CPU 61 determines that the current level CL is less than or equal to the first level L1 (S22: YES), the CPU 61 causes the process to proceed to step S23.

In step S23, the CPU 61 determines whether a notification process has been performed. The determination is made as follows. The CPU 61 repeatedly executes the during-running program P2 until the biological information output device 30 stops running. If, in the repeated execution of the during-running program P2, the notification process, which is described below, in step S27 has been performed even once, the CPU 61 determines that the notification process has been performed. In the repeated execution of the during-running program P2, if step S27 has not been performed even once, the CPU 61 determines that the notification process has not been performed.

If the CPU 61 determines that the notification process has not been performed (S23: NO), the CPU 61 causes the process to proceed to step S27. In step S27, the CPU 61 performs the notification process. The notification process in step S27 is the same as that in step S13 in the initial running program P1. After that, the CPU 61 causes the process to proceed to step S24. In contrast, if the CPU 61 determines that the notification process has been performed in step S23 (S23: YES), the CPU 61 also causes the process to proceed to step S24.

In step S24, the CPU 61 performs a second level-determination process. In the second level-determination process, the CPU 61 determines whether the current level CL acquired in the current-level acquiring process in step S21 is less than or equal to a second level L2. The second level L2 is a predetermined value smaller than that of the first level L1. For example, the second level L2 is determined as the lower limit of the level of the battery 36 with which the biological information output device 30 runs stably. In this embodiment, when it is assumed that the level of the battery 36 in the full-charge state is set to 100%, 5% is set to the second level L2.

If the CPU 61 determines that the current level CL is greater than the second level L2 (S24: NO), the CPU 61 ends the series of processes this time. That is, if the current level CL is less than or equal to the first level L1 and greater than the second level L2, the CPU 61 continues running the biological information output device 30. In contrast, if the CPU 61 determines that the current level CL is less than or equal to the second level L2 (S24: YES), the CPU 61 causes the process to proceed to step S25.

In step S25, the CPU 61 performs a warning process. In the warning process, the CPU 61 outputs a warning signal. The warning signal is a signal for notifying a user that the current level CL is quite low. Specifically, in the warning process, the CPU 61 outputs the warning signal to the small monitor 33. Thus, the small monitor 33 displays a message image indicating that the biological information output device 30 will run just for a short time. The message image is displayed for several seconds to more than ten seconds. After that, the CPU 61 causes the process to proceed to step S26.

In step S26, the CPU 61 performs an end process. The end process in step S26 is the same as that in step S14 in the initial running program P1. That is, in the end process in step S26, the CPU 61 stops the biological information output device 30 after providing, to a user, a prior notice of stopping the biological information output device 30. Thus, the CPU 61 ends the series of processes this time.

### <A Series of Processes Related to Wireless Communication>

When a long-press operation on the switch 34 causes the biological information output device 30 to enter the ON state from the OFF state, that is, when the biological information output device 30 starts running, the CPU 61 executes the wireless communication program P3 only once. The wireless communication program P3 is executed in parallel with the initial running program P1. When running of the biological information output device 30 is stopped due to the initial running program P1, the wireless communication program P3 ends even when it is being executed. At start of running of the biological information output device 30, the biological information output device 30 has not established a wireless communication with a biosensor 20.

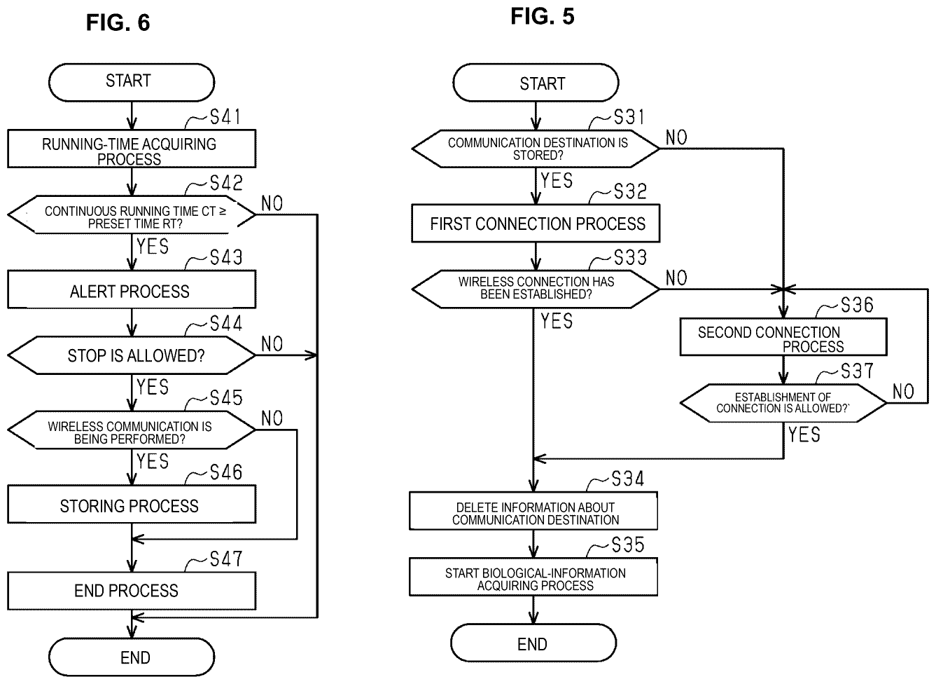

As illustrated in FIG. 5, when the CPU 61 starts the wireless communication program P3, the CPU 61 first performs the process in step S31. In step S31, the CPU 61 determines whether a communication destination is stored in the storage device 64. If the CPU 61 determines that a communication destination is stored in the storage device 64 (S31: YES), the CPU 61 causes the process to proceed to step S32.

In step S32, the CPU 61 performs a first connection process. In the first connection process, the CPU 61 establishes a wireless communication with the biosensor 20 in response to a request from the biosensor 20 stored as a communication destination in the storage device 64. In particular, in the first connection process, the CPU 61 establishes a wireless communication with reception of a request, only from the biosensor 20 stored as a communication destination in the storage device 64, to establish a wireless communication. After that, the CPU 61 causes the process to proceed to step S33.

In step S33, the CPU 61 determines whether a wireless communication has been established with the biosensor 20 stored as a communication destination in the storage device 64. Specifically, if a signal indicating that a wireless communication has been established is received from the biosensor 20 stored as a communication destination in the storage device 64, the CPU 61 determines that a wireless communication has been established. In contrast, if such a signal has not been received, in a predetermined certain time period, from the biosensor 20 stored as a communication destination in the storage device 64, the CPU 61 determines that the wireless communication has not been established. If establishment of the wireless communication has been completed (S33: YES), the CPU 61 causes the process to proceed to step S34.

In step S34, the CPU 61 deletes information about the communication destination stored in the storage device 64. After that, the CPU 61 causes the process to proceed to step S35.

In step S35, the CPU 61 starts a biological-information acquiring process. In the biological-information acquiring process, monitoring results of biological information are acquired from the biosensor 20 through wireless communication. After that, the CPU 61 continues the biological-information acquiring process until the biological information output device 30 stops running. At a stop of the biological information output device 30, the CPU 61 ends the biological-information acquiring process.

If, in step S31, the CPU 61 determines that a communication destination is not stored in the storage device 64 (S31: NO), the CPU 61 causes the process to proceed to step S36. If, in step S33, the CPU 61 determines that a wireless communication has not been established (S33: NO), the CPU 61 causes the process to proceed to step S36.

In step S36, the CPU 61 performs a second connection process. In the second connection process, the CPU 61 allows establishment of a wireless communication with a biosensor 20 which is not stored as a communication destination in the storage device 64. That is, a request, from a biosensor 20 which is not stored as a communication destination, to establish a wireless communication is received. After that, the CPU 61 causes the process to proceed to step S37.

In step S37, the CPU 61 performs a connection approval process. In the connection approval process, under the condition that an establishment request of a biosensor 20 has been received in the second connection process, the CPU 61 outputs a connection-approval request signal. The connection-approval request signal is a signal for a user to select whether establishment of a connection through wireless communication to the biosensor 20 is to be allowed. Specifically, in the connection approval process, the CPU 61 outputs the connection-approval request signal to the small monitor 33. Thus, the small monitor 33 displays a message image for asking whether establishment of a connection to the biosensor 20 is to be allowed. The switch 34 receiving a predetermined operation during display of the message image causes the CPU 61 to obtain, from the user, a signal of permission. If the CPU 61 obtains, from the user, a signal of permission (S37: YES), the CPU 61 establishes a wireless communication with the biosensor 20. After that, the CPU 61 causes the process to proceed to step S34. In contrast, if the switch 34 has not received the predetermined operation, that is, if the CPU 61 has not obtained, from the user, a signal of permission (S37: NO), the CPU 61 causes the process to return to step S36 again.

### <A Series of Processes Related to a Continuous Running Time>

When the biological information output device 30 is in the ON state, the CPU 61 repeatedly performs the continuous running program P4 at predetermined intervals. The continuous running program P4 is performed in parallel with the during-running program P2. After an alert process, which is described below, is performed once, the CPU 61 does not perform the continuous running program P4 until the biological information output device 30 stops running.

As illustrated in FIG. 6, when the CPU 61 starts the continuous running program P4, the CPU 61 first performs the process in step S41. In step S41, the CPU 61 performs a running-time acquiring process. In the running-time acquiring process, the CPU 61 acquires a continuous running time CT from the time point of start of the running of the biological information output device 30 powered by the battery 36. Specifically, the CPU 61 acquires, as the continuous running time CT, a time from the time point at which an operation on the switch 34 causes the biological information output device 30 to enter the power-supply ON state, to the current time at which the running has been continuously performed. After that, the CPU 61 causes the process to proceed to step S42.

In step S42, the CPU 61 performs a running-time determination process. In the running-time determination process, the CPU 61 determines whether the continuous running time CT is longer than or equal to the preset time RT. If the continuous running time CT is shorter than the preset time RT (S42: NO), the CPU 61 ends the series of processes this time. That is, the CPU 61 continues running the biological information output device 30. In contrast, if the continuous running time CT is longer than or equal to the preset time RT (S42: YES), the CPU 61 causes the process to proceed to step S43.

In step S43, the CPU 61 performs the alert process. In the alert process, the CPU 61 outputs an alert signal. The alert signal is a signal for notifying a user that an amount of power for the biological information output device 30 to run for the preset time RT has been consumed. Specifically, in the alert process, the CPU 61 outputs the alert signal to the small monitor 33. Thus, the small monitor 33 displays a message image indicating that the amount of power for the biological information output device 30 to run for the preset time RT has been consumed. This message image is displayed for several seconds to more than ten seconds. After that, the CPU 61 causes the process to proceed to step S44.

In step S44, the CPU 61 performs a stop approval process. In the stop approval process, the CPU 61 first outputs a stop-approval request signal. The stop-approval request signal is a signal for a user to select whether the biological information output device 30 is allowed to stop running. Specifically, in the stop approval process, the CPU 61 outputs the stop-approval request signal to the small monitor 33. Thus, the small monitor 33 displays a message image for asking whether the biological information output device 30 is allowed to stop running. The switch 34 receiving a predetermined operation during display of the message image causes the CPU 61 to obtain, from the user, a signal of permission. If the CPU 61 fails to obtain, from the user, a signal of permission during display of the message image (S44: NO), the CPU 61 ends the series of processes this time. In contrast, if the CPU 61 has obtained, from the user, a signal of permission (S44: YES), the CPU 61 causes the process to proceed to step S45.

In step S45, the CPU 61 determines whether wireless communication is being performed. If wireless communication is not being performed (S45: NO), the CPU 61 causes the process to proceed to step S47. In contrast, if wireless communication is being performed (S45: YES), the CPU 61 causes the process to proceed to step S46.

In step S46, the CPU 61 performs a storing process. In the storing process, the CPU 61 stores the biosensor 20, with which wireless communication is being performed, as a communication destination in the storage device 64. Specifically, the unique ID of the biosensor 20, with which wireless communication is being performed, is stored in the storage device 64. After that, the CPU 61 causes the process to proceed to step S47.

In step S47, the CPU 61 performs an end process. The end process in step S47 is the same as that in step S14 in the initial running program P1. That is, in the end process in step S47, the CPU 61 stops the biological information output device 30 after providing, to a user, a prior notice of stopping the biological information output device 30. Thus, the CPU 61 ends the series of processes this time.

### (Operation of Embodiment)

According to the embodiment, when a user operates the switch 34 to start activation of the biological information output device 30, the control unit 60 starts execution of the initial running program P1. Then, the user may confirm whether a message indicated by the notification signal is present on the small monitor 33. If the message indicated by the notification signal is displayed on the small monitor 33, the user checks the message and confirms that the biological information output device 30 will not run for the preset time RT or longer, that is, for twelve hours or longer in this running. After that, the control unit 60 performs the end process. Thus, the running of the biological information output device 30 is stopped before the running of the biological information output device 30 proceeds in this session.

### (Effect of Embodiment)

(1) According to the embodiment, if it is determined that the initial battery level SBL is less than or equal to the first level L1 in the first level-determination process at start of running, the CPU 61 performs the notification process. In the notification process, the CPU 61 outputs the notification signal. The notification signal is a signal for notifying a user that the biological information output device 30 will not run for the preset time RT. Therefore, the user may confirm, through the notification signal, that the biological information output device 30, which has run, will not run for the preset time RT or longer. This suppresses occurrence of an unexpected stop of the biological information output device 30 in the preset time RT.

(2) According to the embodiment, after the notification process, the CPU 61 further performs the end process. Therefore, in this running of the biological information output device 30, a sudden stop of running of the biological information output device 30 due to battery depletion during running for the preset time RT may be prevented. That is, in the case of occurrence of a potential, unexpected stop during running for the preset time RT, the running of the biological information output device 30 may be stopped at start of the running. The CPU 61 performs the notification process, and then performs the end process. Thus, running of the biological information output device 30 may be stopped in a state predictable by a user.

(3) According to the embodiment, when it is determined that the current level CL is less than or equal to the second level L2 in the second level-determination process, the CPU 61 performs the warning process. In the warning process, the CPU 61 outputs the warning signal. The warning signal is a signal for notifying a user that the current level CL is quite low. Therefore, the user may confirm, through the warning signal, that the biological information output device 30 will run only for a very short time. This may suppress occurrence of the biological information output device 30 entering, after the warning process, a state unpredictable by a user.

(4) According to the embodiment, after the warning process, the CPU 61 further performs the end process. Therefore, running of the biological information output device 30 may be stopped in a state predictable by a user.

(5) According to the embodiment, when the current level CL is less than or equal to the first level L1 and greater than the second level L2, the CPU 61 continues running the biological information output device 30. Therefore, after start of running of the biological information output device 30, even when the current level CL reaches the first level L1 or below, if the current level CL is greater than the second level L2, the running continues. Therefore, the biological information output device 30 may continuously run for a longer time. That is, frequent stops of running of the biological information output device 30 may be avoided.

(6) According to the embodiment, when the continuous running time CT is longer than or equal to the preset time RT, the CPU 61 performs the alert process. In the alert process, the CPU 61 outputs the alert signal. The alert signal is a signal for notifying a user that the battery 36 has consumed power required to run for the preset time RT. Therefore, the user may confirm, through the alert signal, that the running time has exceeded the preset time RT for which running is guaranteed by the fact that the notification process has not been performed. This may suppress occurrence of the biological information output device 30 entering, after the alert process, a state unpredictable by a user.

(7) According to the embodiment, after the alert process, the CPU 61 further performs the end process. Therefore, running of the biological information output device 30 may be stopped in a state predictable by a user.

(8) According to the embodiment, the CPU 61 performs the biological-information acquiring process using wireless communication. Therefore, even if the distance to the biosensor 20, from which biological information is to be monitored, is rather far, the biological information may be obtained. Space around a person, on whom the biosensor 20 is attached, may be easily provided, reducing interference with a surgical operation.

(9) According to the embodiment, when the storage device 64 stores a communication destination, the CPU 61 performs the first connection process. In the first connection process, the CPU 61 establishes a wireless communication only with the biosensor 20 stored as a communication destination by the storage device 64. Therefore, in repeated use of the biological information output device 30 using the same biosensor 20, occurrence of acquiring monitoring results of biological information from a different communication destination may be suppressed.

(10) According to the embodiment, when the continuous running time CT is longer than or equal to the preset time RT, the CPU 61 performs the storing process. In the storing process, the CPU 61 stores a communication destination in the storage device 64. Therefore, even if the continuous running time CT reaches the preset time RT or longer and the end process is then performed, the storage device 64 has stored the communication destination at the next startup. Therefore, a wireless communication may be established with the same communication destination in the next running.

(11) According to the embodiment, when the storage device 64 has not stored a communication destination, the CPU 61 performs the second connection process. In the second connection process, the CPU 61 allows establishment of a wireless communication with a biosensor 20 which is not stored as a communication destination in the storage device 64. Therefore, even when the storage device 64 has not stored a communication destination, a wireless communication may be established to obtain biological information.

(12) According to the embodiment, an operation on the switch 34 causes the power supply of the biological information output device 30 to enter the ON state. At that time, the control unit 60 starts execution of the initial running program P1 in the state in which a user holds the biological information output device 30 in their hand. Therefore, when the notification process is performed in the initial running program P1, the small monitor 33 displays the message while the user holds the biological information output device 30 in their hand. This facilitates the user noticing the message of the notification signal.

### Other Embodiments

The embodiment may be changed as follows for implementation. The embodiment described above and modified examples described below may be implemented by combining these with one another in a scope having no technical contradictions.
The biosensor 20 is not limited to one attached on a monitoring target with the attachment sheet 21 interposed in between. The biosensor 20 may be any as long as it is a device which detects biological information.
The communication system between the biosensor 20 and the biological information output device 30 is not limited to the example of the embodiment described above. The system of wireless communication between them may be ZigBee® or wireless LAN.
In the embodiment, an operation on the switch 34 of the biological information output device 30 indicates input of a signal of permission. However, the biological information output device 30 may have an operable mechanism different from that of the switch 34. For example, the small monitor 33 of the biological information output device 30 may be a touch panel which is operable.
In the embodiment, an operation on the switch 34 causes the power supply of the biological information output device 30 to enter the ON state. Instead of the switch 34, a signal for switching on the power supply may be received through wireless communication.
The biological information output device 30 may have a speaker in addition to or instead of the small monitor 33. In this case, the notification signal, the warning signal, and the alert signal may be outputted from the speaker to output audio messages from the speaker. The notification signal, the warning signal, and the alert signal are not limited to signals for outputting images, and may be signals for outputting light, signals for outputting sound, or signals for stopping biological information displayed on the small monitor 33 continuously.
The biological information output device 30 is not limited to one which outputs monitoring results of biological information through the output terminal 32. For example, the biological information output device 30 may output monitoring results of biological information to the display 40 in the state in which the biological information output device 30 is connected to the display 40 in a wired manner. Alternatively, for example, the biological information output device 30 may output monitoring results of biological information to the display 40 through wireless communication.

### <Control Unit>

The control unit 60 may be formed as circuitry including one or more processors which perform various types of processing according to computer programs (software). The control unit 60 may be formed as one or more dedicated hardware circuits, such as an application-specific integrated circuit (ASIC), which perform at least some types of processing among the various types of processing, or circuitry including a combination of these. A processor includes a CPU and memory such as a RAM and a ROM. The memory stores program codes or instructions which are configured to cause the CPU to perform processing. The memory, that is, a computer-readable medium, includes any available medium accessible by a general-purpose or dedicated computer.
The timing at which the control unit 60 performs the storing process is not limited to the example of the embodiment. When, after establishment of a wireless communication, information about a communication destination is deleted as in step S34 in the embodiment, it is preferable to perform the storing process before the end process. Alternatively, for example, the process in step S34 may be skipped. In this case, the control unit 60 may perform the storing process after the second connection process.
The control unit 60 may skip the stop approval process in step S44 in the series of processes in the continuous running program P4. The control unit 60 may skip the processes in step S45 and step S46. The control unit 60 may skip the end process in step S47.
The control unit 60 may skip execution of the continuous running program P4. The storage device 64 does not necessarily store the continuous running program P4. That is, the control unit 60 may skip execution of the running-time acquiring process in step S41, the process in step S42, and the alert process in step S43.
The control unit 60 may skip execution of the second connection process in step S36. In this case, execution of the connection approval process in step S37 may be also skipped. In this case, for example, the biological information output device 30 may establish a wireless communication only with a predetermined biosensor 20. In this case, for example, any configuration may be employed as long as the storage device 64 always stores the predetermined biosensor 20 as a communication destination.
The control unit 60 may skip execution of the wireless communication program P3. The storage device 64 does not necessarily store the wireless communication program P3. For example, the biological information output device 30 may be connected to the biosensor 20 in a wired manner to obtain biological information from the biosensor 20. Alternatively, for example, the biological information output device 30 may have a sensor which is capable of detecting biological information. In this case, monitoring results of biological information may be obtained from the sensor. In this case, the wireless receiver 35 may be omitted. That is, the biological information output device 30 is not necessarily a device which relays data between the biosensor 20 and the display 40. The biological information output device 30 may have any configuration at least as long as the biological information output device 30 is a device which outputs monitoring results of biological information.
In the series of processes in the during-running program P2, the control unit 60 may perform the notification process in step S27 regardless of the process in step S23. In addition, the control unit 60 may skip the processes in step S22, step S23, and step S27. That is, in the series of processes in the during-running program P2, the current level CL may be compared with the second level L2 without comparison of the current level CL with the first level L1.
In the series of processes in the during-running program P2, when the current level CL is less than or equal to the first level L1 and greater than the second level L2, the control unit 60 may stop running the biological information output device 30. For example, even when the current level CL is less than or equal to the first level L1 and greater than the second level L2, in some cases, the end process in step S47 in the continuous running program P4 stops running the biological information output device 30. In addition, for example, after execution of the alert process in step S43, when the current level CL reaches the first level L1 or below, even if the current level CL is greater than the second level L2, the control unit 60 may perform the end process after the notification process in step S27.
In the series of processes in the during-running program P2, the control unit 60 may skip the processes in step S24 and step S25.
In the series of processes in the during-running program P2, the control unit 60 may skip the end process in step S26. In this case, for example, when the level of the battery 36 reaches zero after the warning process in step S25, running of the biological information output device 30 may be stopped due to battery depletion. Even in this case, battery depletion may occur in a state predictable by a user due to the warning process.
The control unit 60 may skip execution of the during-running program P2. The storage device 64 does not necessarily store the during-running program P2. The control unit 60 may perform at least the initial running program P1 among the four programs.
In the end process in step S14 in the initial running program P1, the control unit 60 may end running of the biological information output device 30 without outputting the prior-notice-of-stop signal. This point is substantially the same in the end processes in other programs.
In the series of processes in the initial running program P1, the control unit 60 may skip the end process in step S14. In the series of processes in the initial running program P1, at least the following operation may be performed: when it is determined that the initial battery level SBL is less than or equal to the first level L1, the control unit 60 performs the notification process.
In a device as described in Japanese Unexamined Patent Application Publication No. 2015-141080, continuous running for the preset time RT or longer may cause the device to stop at an unexpected time. In the embodiment, the control unit 60 performs the running-time acquiring process and the alert process. Thus, a user may confirm, on the basis of the alert signal, that the running time has exceeded the preset time RT for which running is guaranteed by the fact that the notification process has not been performed. This may suppress occurrence of the biological information output device 30 entering, after the alert process, a state unexpected by the user. Thus, in view of being able to confirm that the running time has exceeded the preset time RT, the control unit 60 does not necessarily perform the initial-battery-level acquiring process, the first level-determination process, and the notification process.

### APPENDIX

Technical idea derived from the embodiment and modified examples will be described below.
<1> A biological information output device comprising: a control unit that controls running and stopping of the biological information output device which runs on power supply from a battery and which outputs biological information, wherein the control unit performs an initial-battery-level acquiring process of acquiring an initial battery level which is the level of the battery at start of the running of the biological information output device, a first level-determination process of determining whether the initial battery level is less than or equal to a first level, the first level being a level required for the biological information output device to run just for a preset time which is predetermined, and a notification process of, when it is determined that the initial battery level is less than or equal to the first level in the first level-determination process, outputting a notification signal for notifying a user that the running will not continue for the preset time.
<2> The biological information output device according to <1>, wherein the control unit further performs an end process of, after the notification process, ending the running of the biological information output device running on the power supply of the battery.
<3> The biological information output device according to <1> or <2>, wherein the control unit further performs a current-level acquiring process of acquiring a current level which is the current level of the battery, a second level-determination process of, during running of the biological information output device, determining whether the current level is less than or equal to a second level, the second level being predetermined as a value smaller than the first level, and a warning process of outputting a warning signal for notifying a user when it is determined that the current level is less than or equal to the second level in the second level-determination process.
<4> The biological information output device according to <3>, wherein the control unit further performs an end process of, after the warning process, ending the running of the biological information output device running on power supply from the battery.
<5> The biological information output device according to <3> or <4>, wherein, when the current level is less than or equal to the first level and greater than the second level, the control unit continues running the biological information output device.
<6> The biological information output device according to any one of <1> to <5>, wherein the control unit further performs a running-time acquiring process of acquiring a continuous running time since start of the battery-powered running, and an alert process of, when the continuous running time is longer than or equal to the preset time, outputting a signal for notifying a user that power required for running for the preset time is consumed.
<7> The biological information output device according to <6>, wherein the control unit further performs an end process of, after the alert process, ending the running of the biological information output device running on power supply from the battery.
<8> The biological information output device according to any one of <1> to <7>, wherein the control unit further performs a biological-information acquiring process of acquiring, through wireless communication, the biological information from a biosensor which monitors the biological information.
<9> The biological information output device according to <8>, wherein the control unit has a storage device, wherein the control unit further performs a storing process of storing the biosensor as a communication destination in the storage device, the biosensor being a biosensor with which the wireless communication is performed, and a first connection process of, when a wireless communication has not been established, establishing a wireless communication with the biosensor in response to a request from the biosensor, and wherein, in the first connection process, the control unit establishes the wireless communication only with the biosensor stored as the communication destination in the storage device.
<10> The biological information output device according to <9>, wherein the control unit further performs a running-time acquiring process of acquiring a continuous running time since start of the battery-powered running, and wherein the control unit performs the storing process when the continuous running time acquired in the running-time acquiring process is longer than or equal to the preset time.
<11> The biological information output device according to <9> or <10>, wherein the control unit further performs a second connection process of, when a wireless communication with the biosensor stored as the communication destination in the storage device has not been established, allowing establishment of a wireless communication with the biosensor which is not stored as the communication destination in the storage device.
<12> A biological information output device comprising: a control unit that controls running and stopping of the biological information output device which runs on power supply from a battery and which outputs biological information, wherein the control unit performs a running-time acquiring process of acquiring a continuous running time since start of the battery-powered running, and an alert process of, when the continuous running time is longer than or equal to a preset time which is predetermined, outputting a signal for notifying a user that power required for running for the preset time is consumed.
10 biological information acquiring system
20 biosensor
21 attachment sheet
30 biological information output device
31 body case
32 output terminal
33 small monitor
34 switch
35 wireless receiver
36 battery
40 display
60 control unit
61 CPU
62 peripheral circuitry
63 ROM
64 storage device
CL current level
CT continuous running time
L1 first level
L2 second level
RT preset time
SBL initial battery level

1. A biological information output device comprising:
a controller that controls running and stopping of the biological information output device which runs on power supply from a battery and which outputs biological information,
wherein the controller is configured to:
acquire an initial battery level which is a level of the battery at a start of the running of the biological information output device,
determine whether the initial battery level is less than or equal to a first level, the first level being a level required for the biological information output device to run for a preset time, and
when it is determined that the initial battery level is less than or equal to the first level, output a notification signal that notifies a user that the running will not continue for the preset time.

2. The biological information output device according to claim 1, wherein the controller is further configured to:
after outputting the notification signal, end the running of the biological information output device running on the power supply of the battery.

3. The biological information output device according to claim 1, wherein the controller is further configured to:
acquire a current level of the battery,
during running of the biological information output device, determine whether the current level is less than or equal to a second level, the second level being less than the first level, and
output a warning signal that notifies the user when it is determined that the current level is less than or equal to the second level.

4. The biological information output device according to claim 3, wherein the controller is further configured to:
after outputting the warning signal, end the running of the biological information output device running on power supply from the battery.

5. The biological information output device according to claim 3, wherein, when the current level is less than or equal to the first level and greater than the second level, the controller is configured to continue running the biological information output device.

6. The biological information output device according to claim 1, wherein the controller is further configured to:
acquire a continuous running time since start of the battery-powered running, and
when the continuous running time is longer than or equal to the preset time, output a signal that notifies the user that power required for running for the preset time is consumed.

7. The biological information output device according to claim 6, wherein the controller is further configured to:
after outputting the signal, end the running of the biological information output device running on power supply from the battery.

8. The biological information output device according to claim 1, wherein the controller is further configured to:
acquire, through wireless communication, the biological information from a biosensor.

9. The biological information output device according to claim 8,
wherein the controller comprises memory,
wherein the controller is further configured to:
store the biosensor as a communication destination in the memory, the biosensor being a biosensor with which the wireless communication is performed, and
when wireless communication has not been established, establish wireless communication with the biosensor in response to a request from the biosensor, and
wherein the controller is configured to establish wireless communication only with the biosensor stored as the communication destination in the storage device.

10. The biological information output device according to claim 9,
wherein the controller is further configured to:
acquire a continuous running time since start of the battery-powered running, and
wherein the controller is configured to store the biosensor as the communication destination when the continuous running time acquired in the running-time acquiring process is longer than or equal to the preset time.

11. The biological information output device according to claim 9, wherein the controller is further configured to:
when wireless communication with the biosensor stored as the communication destination has not been established, allow establishment of wireless communication with a biosensor which is not stored as the communication destination.
